(19) United States
(12) Plant Patent Application Publication
(10) Pub. No.: US 2016/0073564 P1
(43) Pub. Date: Mar. 10, 2016

(54) Medinilla hybrid 'Arosa'

(50) ;

(71) Applicants: Elly Bak, Rijsenhout (NL); Nicolaas Steur, Oude Niedorp (NL)

(72) Inventors: Elly Bak, Rijsenhout (NL); Nicolaas Steur, Oude Niedorp (NL)

(73) Assignee: Corn Bak B.V., Assendelft (NL)

(21) Appl. No.: 14/121,492

(22) Filed: Sep. 10, 2014

(51) Int. Cl. A01H 5/00

(52) U.S. Cl. PLT/226

(57) ABSTRACT

A new and distinct Medinilla hybrid plant named ‘AROSA’, characterized by an erect shrub plant, measuring about 30 cm in total height (when first flowering) and about 40 cm in diameter; green colored leaves, measuring about 13-18 cm in length and bout 7 cm in width; pendulous panicle, measuring about 20 cm in length and about 10 cm in diameter, with red-purple (RHS 65D) flowers.

### LATIN NAME OF THE GENUS AND SPECIES

Medinilla hybrid

### VARIETY DENOMINATION

‘AROSA’

### BACKGROUND OF THE INVENTION

The present invention relates to a new and distinct hybrid cultivar of Medinilla plant, botanically known as Medinilla hybrid, hereinafter referred to by the cultivar name ‘AROSA’. The genus of Medinilla belongs to the Melastomataceae family.

The new Medinilla ‘AROSA’ is a product of a planned breeding program conducted by the inventors, Elly Bak and Nico D. M. Steur, in Assendelft, The Netherlands. The objective of the breeding program is to develop a new Medinilla variety with a good growing habit and attractive inflorescence.

The new Medinilla ‘AROSA’ is the result of some generations of selection in a Medinilla hybrid. The female or seed parent of the hybrid is proprietary, unpatented Medinilla selection identified by code 82984514. The female or seed parent of the hybrid is proprietary, unpatented Medinilla selection identified by code 82981030. The crossing resulting in Medinilla ‘AROSA’ was made during 2009 and with the plant flowered for the first time in 2011 in Assendelft, the Netherlands.

Asexual reproduction of the new Medinilla hybrid ‘AROSA’ performed by vegetative means by cuttings was first performed in 2011, in Assendelft, the Netherlands. The first ‘AROSA’ plants propagated through the use of cuttings flowered in 2012 in Assendelft, the Netherlands, and have demonstrated that the combination of characteristics herein disclosed for the new cultivar are firmly fixed and retained through successive generations of asexual reproduction. The new cultivar reproduces true-to-type.

### BRIEF DESCRIPTION OF THE INVENTION

The following traits have been repeatedly observed and are determined to be unique characteristics of ‘AROSA’ which in combination distinguish the Medinilla hybrid as a new and distinct cultivar:

1. Solid growth habit;

2. Erect shrub plant, measuring about 30 cm in height (above the pot when first flowering);

3. Green colored leaves, measuring about 13-18 cm in length and about 7 cm in width;

4. Pendulous panicles with red-purple (RHS 65D) colored flowers;

Of the many commercials cultivars known to the present inventors, the most similar in comparison to the new Medinilla ‘AROSA’ is the Medinilla ‘MAGIC’ application Ser. No. 13/694,587. Plants of the new Medinilla ‘AROSA’ differ from plants of the Medinilla ‘MAGIC’ primarily in leaf size. Plants of ‘MAGIC’ have smaller leaves than plants of ‘AROSA’.

### BRIEF DESCRIPTION OF THE PHOTOGRAPHS

The accompanying photograph illustrates the overall appearance of the new Medinilla ‘AROSA’ showing the colors as true as is reasonably possible with colored reproductions of this type. Colors in the photograph may differ slightly from the color values cited in the detailed botanical description which accurately describes the color of ‘AROSA’.

FIG. 1 shows a side view perspective of a typical potted flowering plant of ‘AROSA’ at about 50 weeks from potting.

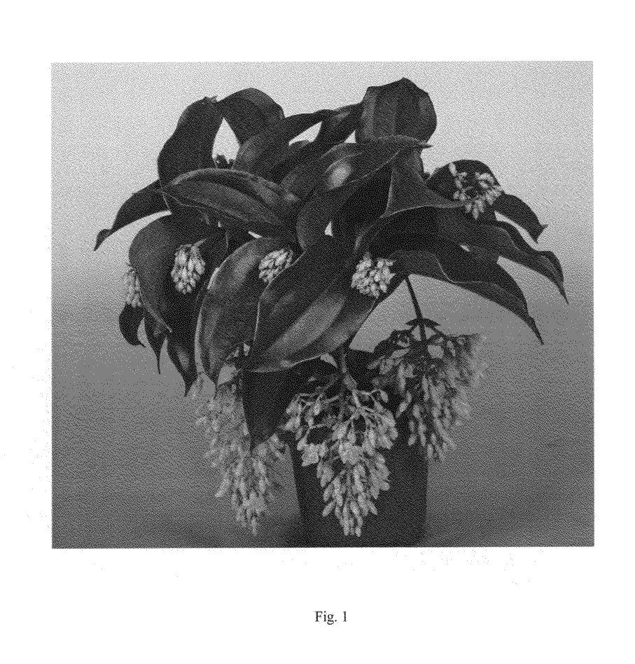

### DETAILED BOTANICAL DESCRIPTION

‘AROSA’ has not been observed under all possible environmental conditions. The phenotype of the new cultivar may vary with the variations in environment such as temperature, light intensity, day length, fertilizers and humidity without any change in the genotype of the plant.

The aforementioned photograph, together with the following observations, measurements and values describes the new Medinilla ‘AROSA’ as grown in a greenhouse in Assendelft, the Netherlands, under conditions which closely approximate those generally used in commercial practice.

Color references are made to The Royal Horticultural Society Colour Chart (R.H.S.), 2001 Edition, except where general colors of ordinary significance are used. Color values were taken under daylight conditions in a greenhouse in Assendelft, the Netherlands. The age of the plants of ‘AROSA’ described is about 50 weeks from potting.
Botanical: Medinilla hybrid
Propagation: Vegetative cuttings
Plant:
   General appearance and form.—Height: About 30 cm Diameter: About 40 cm Shape: Semi-upright fleshy plant with pendulous flowers.
   Growth habit.—Semi-upright.
   Plant vigor.—Moderate to vigorous.
   Flowering season.—from April till October.
   Cold tolerance.—frost tender, temperatures below 5 degree C. may damage plants.
   Fragrance.—none.
   Stem.—Shape: Square and winged. Wings approximately 2 mm wide and 8 mm long Internode length: Average range around 4 to 7 cm. Color: Near RHS 143A.
Foliage:
   Quantity.—About 30.
   Arrange.—Opposite.
   Size.—Length: About 13 to 18 cm. Width: About 7 cm.
   Overall shape.—broadly ovate.
   Apex shape.—cuspidate.
   Base Shape.—acute.
   Margin.—Entire, slightly undulate.
   Texture.—Glabrous.
   Color.—Leaf color can vary somewhat depending on growing conditions. Upper surface: Glossy green, closest to RHS 139A Lower surface: Glossy, closest to RHS 137C.
   Venation.—Parallel, sunken, the venation is slightly below the surface of the rest of the leaf. Upper surface: Near RHS Yellow-Green 144B Lower surface: Near RHS Yellow-Green 146B.
Inflorescence:
   Shape.—Pendulous panicles.
   Amount.—about 10 per plant (depending on the size of the plant).
   Size.—Length: About 20 cm Width: About 10 cm.
   Corolla.—Number: about 190 per panicle Length: About 20 mm Width: About 10 mm.
   Petals.—Number: 4 or 5 per flower Length: 14 mm Width: 8 mm Overall Shape: obovate, asymmetrical Apex Shape: obtuse Color: Upper surface and lower: white, closest to RHS 155B.
   Sepals.—Fused together, enclosing the ovary. Less than 8mm Color: red-purple, closest to RHS 65D.
   Bracts.—Quantity: 2 per crown Arrangement: Verticillate Size: Length: about 1-18 mm Width: less than 1-13 mm Overall shape: ovate Color: Near RHS Red-Purple 64B and Yellow-Green 144B.
   Bud.—Size: Length: about 12 mm Width: about 4 mm Shape: Ovate Color: Near RHS Red-Purple 62D.
   Peduncle.—Length: About 7.0 cm. Diameter: About 0.3 cm. Color: RHS Yellow-Green 144B, over time changing to Red-Purple 64A Texture: glabrous.
   Pedicel.—Length: Average range approximately 0.5cm to 20 cm. Shorter pedicels occurring with terminal flowers, longer towards base of inflorescence. Diameter: About 0.10 cm. Texture: Glabrous Color: RHS Red-Purple 65A.
Reproductive organs:
   Androecium.—Stamen: Number: 8 per flower Length: 10 mm Diameter: 1 mm Color: yellow, with a white base (too small to qualify RHS value) Anther: Length: 8 mm Color: Violet, closest to RHS 86B, with a yellow base (base too small to qualify RHS value) Pollen: not visible Gynoecium: Pistil: Number: 1 per flower Length: 19 mm Stigma: Width: less than 1 mm Color: white, too small to qualify RHS value Style: Length: 16 mm Color: white, closest to RHS 155B Ovary: Shape: round Position: inferior Length: 3 mm Diameter: 3 mm Color: white, closest to RHS 155C.
Seeds:
   Amount.—numerous.
   Shape.—obovate glabrous.
   Size.—<1 mm.
   Color at maturity.—grey-orange, closest to RHS 165B.
Fruit:
   Type.—berry.
   Shape.—round.
   Texture.—smooth.
   Size.—0.5-1 cm.
   Color at maturity.—grey-purple closest to 187A.
Disease/pest resistance: Neither resistance nor susceptibility to the normal diseases and pests of Medinilla have been observed.

What is claimed is:

1. A new and distinct Medinilla plant name ‘AROSA’, as illustrated and described herein.
